(19) United States
(12) Patent Application Publication
(10) Pub. No.: US 2012/0251505 A1
(43) Pub. Date: Oct. 4, 2012

(54) HUMAN LYMPHOID TISSUE INDUCER (LTi) CELL COMPOSITIONS AND METHODS OF USE

(75) Inventors: Carl F. Ware, Solana Beach, CA (US); Vasileios Bekiaris, San Diego, CA (US)

(73) Assignee: La Jolla Institute for Allergy and Immunology, La Jolla, CA (US)

(21) Appl. No.: 13/394,010

(22) PCT Filed: Sep. 3, 2010
(86) PCT No.: PCT/US2010/047823

§ 371 Date: Jun. 12, 2012

Related U.S. Application Data
(60) Provisional application No. 61/240,094, filed on Sep. 4, 2009.

(51) Int. Cl. A61K 35/26; C12Q 1/04; G01N 33/569; A61P 31/18; C12Q 1/02; C40B 30/06; A61P 31/22; C12N 5/0789; C12Q 1/68

(52) U.S. Cl. 424/93.7; 435/372; 435/34; 435/7.21; 435/6.12; 435/29; 506/10

(57) ABSTRACT

The invention provides human lymphoid tissue inducer (LTi) cells, methods of producing human lymphoid tissue inducer (LTi) cells, and methods of using human lymphoid tissue inducer (LTi) cells. Such methods include treatment of a subject that would benefit from human lymphoid tissue inducer (LTi) cells, for example, an immunocompromised or immunosuppressed subject.

### RELATED APPLICATIONS

This application claims the benefit of priority of application Ser. No. 61/240,094, filed Sep. 4, 2009, which is expressly incorporated herein by reference in its entirety.

### STATEMENT AS TO FEDERALLY SPONSORED RESEARCH

This invention was made with Government support under Grant No. AI033068 awarded by the National Institutes of Health. The Government has certain rights in this invention.

### TECHNICAL FIELD

The invention relates to human lymphoid tissue inducer (LTi) cells, methods of producing human lymphoid tissue inducer (LTi) cells, and methods of using human lymphoid tissue inducer (LTi) cells, including treatment of a subject that would benefit from human lymphoid tissue inducer (LTi) cells such as an immunocompromised or immunosuppressed subject.

### INTRODUCTION

The lymphotoxin-(LT)αβ-LTβ receptor (LTβR) system (Crowe, et al., Science (1994) 264:707) functions as a key signaling pathway involved in the embryonic development and maintenance of the lymphoid organs. Mice deficient in Ltα, Ltβ or Ltβr fail to form lymph nodes and Peyer's patches, and display distorted architecture in the spleen (De Togni, et al., Science (1994) 264:703; Banks, et al., J Immunol (1995) 155:1685; Futterer, et al., Immunity (1998) 9:59). Indeed, pharmacological blockade of LTαβ signaling with a soluble decoy of LTβR-Fc in pregnant mice caused the loss of secondary lymphoid organs in the offspring, phenotypically replicating the genetic knockouts (Rennert, et al., J Exp Med (1996) 184:1999). The LTβR pathway also functions in the adult as an important immune regulatory pathway controlling T cell and B cell responses by providing differentiative signals in stromal cells and myeloid cells, particularly antigen presenting dendritic cells (Browning, et al., Immunol Rev (2008) 223:202; Gommerman, et al., Nat Rev Immunol (2003) 3:642; Ware, Immunol Rev (2008) 223:186).

The LTβR pathway is recognized as a component of a larger signalling network involving TNF, LIGHT and their specific receptors, TNFR1, TNFR2 and herpesvirus entry mediator (HVEM) (Ware, Annu Rev Immonol (2005) 23:787). The LTβR can be activated by both LTαβ and LIGHT. The role of LIGHT during the embryonic period of lymphoid organogenesis is apparently minor, with effects limited to mesenteric nodes as defined in Light^{-/-} mice (Scheu, et al., J Exp Med (2002) 195:1613). LIGHT also engages HVEM (Mauri, et al., Immunity (1998) 8:21), and HVEM binds the Igsuperfamily members, BTLA (Sedy, et al., Nat Immunol (2005) 6:90) and CD160 (Cai, et al., Nat Immunol (2008) 9:176), and mediates inhibitory signaling limiting T and B lymphocyte and dendritic cell proliferation (Kaye, Nat Immunol (2008) 9:122; Murphy, et al., Nat Rev Immunol (2006) 6:671; Cai, et al., Immunol Rev (2009) 229:244). TNFR1 pathway contributes with LTβR in formation of intestinal Peyer's patches (Rutschmann, et al., J Immunol (2006) 176:7525; Neumann, et al., J Exp Med (1996) 184:259).

The possibility of a specific cell type that induces lymphoid organogenesis, termed the lymphoid tissue inducer (LTi), came from the observation that mice deficient in T and B cells (RAG^{-/-} or SCID) possessed lymph node structures, but were unpopulated with lymphocytes. In the developing embryo, the early LN anlagen contain population(s) of hematopoietically derived cells that express CD4 but not CD3 and other lymphocyte lineage markers (Mebius, et al., Immunity (1997) 7:493). These CD4+CD3− cells expressed high levels of the α-chain of the interleukin (IL)-7 receptor (IL-7Rα) and surface LTβ (Mebius, et al., Immunity (1997) 7:493). CD4+CD3− population was able to differentiate into NK cells, and antigen presenting DC cells, but not T or B cells (Mebius, et al., Immunity (1997) 7:493; Yoshida, et al., J Immunol (2001) 167:2511). These observations suggested that CD4+CD3− cells were capable of inducing the formation of lymphoid organs through the action of LTαβ-LTβR pathway (Mebius, et al., Immunity (1997) 7:493). Supportive evidence for the role of LTi cells in LN development was provided when it was reported that deficiency in the transcription regulators Id2 or RORγt (encoded by Rorc) affected the survival and development of CD4+CD3− cells and loss of lymph nodes (Yokota, et al., Nature (1999) 397:702; Sun, et al., Science (2000) 288:2369; Eberl, et al., Nat Immunol (2004) 5:64). Importantly, adoptive transfer of CD4+CD3− cells in neonates restored Peyer's patch organogenesis (Finke et al., Immunity (2002) 17:363). It was subsequently reported that the CD4+CD3− lymphoid tissue inducer (LTi) cells activate LTβR expressing embryonic stromal cells within the forming anlagen via expression of LTαβ. The activation of LTβR in embryonic stromal cells induces the expression of homeostatic chemokines (CCL21, CXCL13) and adhesion molecules that coalesce B, T and dendritic cells (DC). IL-7 produced by the stromal cells upregulates LTαβ on LTi cells sustaining a signalling loop between the two cell types (Mebius, Nat Rev Immunol (2003) 3:292). The development of Peyer's patches is dependent on IL-7, whereas RANK ligand is necessary for lymph node development, although both of these cytokines induce surface LTαβ (Yoshida, et al., Immunity (2002) 17:823).

LTi cells were previously considered an embryonic population, but may populate adult lymphoid tissue and are extremely rare. In the mouse there exists a population of LTi-like cells that persist through adulthood (Kim, et al., Immunity (2003) 18:643). Adult LTi cells exhibit a very similar phenotype with embryonic LTi (Kim, et al., J Immunol (2006) 177:3074) and retain their ability to induce formation of secondary lymphoid organs. The adoptive transfer of adult LTi cells into LTα^{-/-} mice was able to restore splenic B and T cell segregation and expression of homeostatic chemokines (Kim, et al., Blood (2007) 109:1602). In addition, adult LTi cells were sufficient to induce the formation and organization of embryonic spleen (Glanville, et al., Eur J Immunol (2009) 39:280). A striking feature of adult LTi cells is their expression of OX40-ligand and CD30-ligand (Kim, et al., Immunity (2003) 18:643), both of which have been implicated in promoting differentiation of memory T cells (Croft, Nat Rev Immunol (2003) 3:609). Accordingly, OX40 and CD30 signals from adult LTi to effector CD4 T cells were necessary for sustained memory antibody responses (Kim, et al., Immunity (2003) 18:643; Gaspal, et al., J Immunol (2005) 174:3891). Furthermore, close interactions of CD30-ligand expressing LTi cells with T zone stroma appears important for stromal cell maturation and the ability to segregate B from T cells (Bekiaris, et al., J Immunol (2007) 179:7535), whereas in synergy with LTαβ, LTi cells sustain the expression of the T cell specific homeostatic chemokine, CCL21 (Bekiaris, et al., J Immunol (2009) 182:4771).

LTi cells in the adult may be mediators of inflammatory processes. LTi cells may induce tertiary lymphoid structures in the context of inflammatory signals (Cupedo, et al., Immunity (2004) 21:655). Similarly, disruption of homeostatic signals during embryonic life that lead to accelerated LTi survival results in multiple ectopic lymphoid structures in the adult (Meier, et al., Immunity (2007) 26:643), linking LTi to inflammation (Cupedo, et al., Semin Immunol (2003) 15:243). In adaptive immunity, the LTαβ-LTβR pathway has the unique capacity of inducing proliferation of CD8α-DC subsets in lymphoid organs (Kabashima, et al., Immunity (2005) 22:439; De Trez, et al., J Immunol (2008) 180:238).

LTi cells are implicated in host defense and homeostasis. Following virus-induced lymphoid organ disruption, LTi can restore B and T cell organization (Scandella, et al., Nat Immunol (2008) 9:667). Moreover, in response to fungal antigens LTi rapidly produce IL-17 and IL-22, both of which are implicated in protective immunity (Takatori, et al., J Exp Med (2009) 206:35).

Although suspected for some time, only this year was the first report of a human LTi-like cell reported (Cupedo, et al., Nat Immunol (2009) 10:66). In human embryonic mesentery, a CD4 negative population with LTi activity (LTαβ expression) and phenotype (IL-7Ra, Rorc) (Cupedo, et al., Nat Immunol (2009) 10:66) appears to be a precursor of gut-restricted NK cells (Cupedo, et al., Nat Immunol (2009) 10:66; Luci, et al., Nat Immunol (2009) 10:75; Sanos, et al., Nat Immunol (2009) 10(1):83, epub 2008 Nov. 23; Satoh-Takayama, et al., Immunity (2008) 29:958). The lack of CD4 expression by these human cells correlates to a first order with the fact that LTi sub-populations were identified in both the mouse embryo and adult based on the expression or absence of CD4 (Kim, et al., Immunology (2008) 124:166). A CD4+ LTi cell in human adult or embryo has yet to be identified. In part, the underlying problem may be that these cells are extremely rare and their phenotypic markers common to other lineages, thus human LTi cells are not as accessible as LTi cells are in the mouse, where defined genetic deficiencies have been essential for their characterization. Thus, there is a need to identify cell populations of similar phenotype and function.

### DESCRIPTION OF DRAWINGS

FIG. 1 shows identification of lymphoid tissue inducer (LTi) cells in human adult peripheral blood. (A) Flow chart of the method for identifying putative LTi cells. PBMC were stained with biotin-conjugated anti-human CD3β and CD19. Depletion of CD3+ and CD19+ cells was achieved with streptavidin magnetic particles using the BD IMag technique according to the manufacturer's instructions. A second round of CD14 and CD3β depletion using the same method followed this procedure. The remaining cells were then cultured for 2 days at a density of 10⁶/ml in the presence or absence of recombinant human IL-7 (10 ng/ml). (B) At day 2 after culture, depleted cells were stained for CD3ε, CD19, CD14, CD11c, and CD56 (Lin+). Total PBMC were stained as positive controls. Linage negative (Lin−) cells (red gate) were analyzed for expression of CD4, IL-7Rα, and LTβR ligands in the presence or absence of IL-7. (C) Dot plots: CD4 and IL-7Rα staining of Lin− gated cells with or without IL-7 (results are representative of six studies from different donors with similar results). Histograms: LTβR ligand staining using human LTβR-Fc in the Lin−CD4+ cells with or without IL-7 (control stain was similar to cultures with IL-7) (results are representative of two studies with cells from different donors). (D) Quantitative real-time PCR for Lta, Ltb, and Light in Lin−CD4+ sorted cells in the presence or absence of IL-7 (n.d.=not detected). mRNAexpression was normalized to the house-keeping gene L32 (a value of 1 means equal expression of L32 and target gene). (E) Graphic representation of the proportion of Lin−CD4+ putative LTi cells within the CD3/CD19/CD14 depleted PBMC population after 2 days of culture with or without IL-7 (results are from 5 different studies).

FIG. 2 shows expansion of mouse adult LTi cells in vitro. (A) Flow chart of the method used to culture mouse adult LTi cells; human embryonic kidney-293T (HEK-293T, ATCC CRL-11268) cells were grown at 1×10⁵ in 2 ml in D10 (DMEM+10% FCS). Rag1−/− splenocytes were isolated and depleted of DX5+ and CD11b+ cells by immune depletion with BD IMag technique. The cells (˜1.5×10⁶) were cultured for 2 days in either D10, medium from HEK-293T cells for 2 days, or in D10 in the presence of HEK-293T cells. (B) After 2 days of culture, cells were stained for B220, CD11c, NKp46, CD11b, Gr-1 to define hematopoietic lineages (Lin). Lineage negative (Lin−) cells (red gate) were analyzed further for their expression of CD4 and IL-7Rα. The number of LTi cells recovered from each culture is indicated (Representative of 2 studies).

FIG. 3 shows the HEK-293T cell line sustains adult LTi cells. Rag1^{-/-} splenocytes were depleted of DX5+ and CD11b+ cells and the Lin^{neg} cells were placed in culture with DMEM (10% FBS) only or with HEK-293T cells (1×10⁵-2×10⁵). (A) Cells were analyzed for CD4 and IL-7Rα immediately (d0) or after 2 days. In dot plots, red colored dots correspond to Lin^{neg} that were overlayed in against the CD4/IL-7Rα staining profile. The numbers of LTi cells (CD4+Lin^{neg}) recovered from each culture condition using the same staining and gating strategy (mean sem, n=5). (B) Cells that had been in culture for 32 hrs were re-cultured with HEK-293T cells for a further 16 hrs at which point the cells were analyzed by flow cytometry.

FIG. 4 shows HEK-293T cells do not induce differentiation of LTi progenitors. Rag1−/− splenocytes were stained as shown and the indicated gated populations were FACS-sorted and co-cultured with HEK-293T cells. 2 days later each culture was analyzed for the presence of CD4^{hi}IL-7Rα cells.

FIG. 5 shows re-evaluation of putative human CD4+Lin^{neg} after HEK-293T cell cocultures. CD3ε, CD19, CD14, CD56 depleted human blood cells were cocultured with HEK-293T in the presence or absence of IL-7. Two days later, the cultures were stained for CD56, CD11c, CD11b, CD19, CD14, CD3ε, CD4, IL-7Rα, and LTβR-Fc.

### SUMMARY

The invention provides mammalian lymphoid tissue inducer (LTi) cells, and cultures of lymphoid tissue inducer (LTi) cells, methods for producing lymphoid tissue inducer (LTi) cells, and methods for maintaining survival, growth, proliferation, expanding or increasing numbers of lymphoid tissue inducer (LTi) cells, including mammalian adult, human and mouse LTi cells. Human peripheral blood is one source of CD4+Lin^{neg} cells, which are highly IL-7 responsive. The presence of CD4⁺Lin^{neg}IL-7Rα⁺ cells that express ligands for the LTβR and CD30 in response to IL-7, and Rorc, a key transcription factor controlling LTi lineage, is consistent with designation as human adult LTi cells.

In accordance with the invention, isolated or purified lymphoid tissue inducer (LTi) cells, as well as cultures of lymphoid tissue inducer (LTi) cells are provided. LTi cells are typically characterized by one or more of expression of: CD4, interleukin (IL)-7 receptor alpha, LTalpha, LTbeta, CD30 ligand, CC chemokine receptor 7 (CCR7), CD45, Integrin β7, inhibitor of DNA binding 2 (Id2) or retinoid-related orphan receptor gamma (Rorc) protein or mRNA. LTi cells are also optionally characterized by absence of expression of one or more of: IL-17A, IL-22 CD3, CD14, CD16, CD19, CD56, natural killer (NK)-activating receptor NKp44, CD11c, CD11b, human leukocyte antigen DR-1 (HLA-DR), T cell receptor (TCR)-alpha and -beta chains (TCR alpha/beta), recombination activating gene 1 (Rag1), OX40-ligand, LIGHT (lymphotoxin-like, exhibits inducible expression, and competes with HSV glycoprotein D for HVEM, expressed by T lymphocytes), CC chemokine receptor 6 (CCR6), CXC chemokine receptor 5 (CXCR5), B cell marker B220, CD11b or IFN-gamma, protein or mRNA expression. LTi cells are further optionally characterized by one or more of: inducing or stimulating T cells to produce CC chemokine ligand 21 (CCL21), CC chemokine ligand 19 (CCL19), or CXC chemokine ligand 13 (CXCL13), or inducing or stimulating proliferation or differentiation of myeloid dendritic cells (DC). LTi cells are additionally optionally characterized by an ability to migrate to lymph nodes, Peyer's patches, or spleen white pulp; contributing to or participating in restoration or formation of lymph nodes, Peyer's patches, or spleen white pulp; stimulating or inducing stromal cells to upregulate an adhesion molecule; or ability to differentiate into natural killer (NK) cells, antigen presenting cells (APC), or dendritic cells (DC), and an inability to differentiate into T cells or B cells.

LTi cells include mammalian (e.g., human, mouse, etc.) cells. LTi cells are typically adult cells, for example, a mammal or a human from about 1 hour up to 100 years in age.

Cultures of LTi cells can be viably maintained or sustained, or are proliferating or increasing in numbers. Cultures of LTi cells can include other cells or conditioned medium. In particular embodiments, LTi cells are co-cultured with human embryonic kidney-293T (HEK-293T, ATCC CRL-11268), NHDF (normal human dermal fibroblasts) or HT-29 (colonic epithelial tumor cell line, ATCC HTB-38) cells, or human embryonic kidney-293T (HEK-293T, ATCC CRL-11268), NHDF (normal human dermal fibroblasts) or HT-29 (colonic epithelial tumor cell line, ATCC HTB-38) cell conditioned medium.

In accordance with the invention, methods of producing human lymphoid tissue inducer (LTi) cells are provided. Such methods include one or more of the following selection/screening steps in no particular order: removing or depleting cells expressing CD3 and CD19 from blood cells of a human donor thereby producing a cell population depleted of cells expressing CD3 and CD19; removing or depleting cells expressing CD41 and CD14 from the cell population thereby producing a cell population depleted of cells expressing CD41 and CD14; contacting the cell population depleted of cells expressing CD3, CD19, CD41 and CD14 with IL-7; co-culturing the cell population with human embryonic kidney-293T (HEK-293T, ATCC CRL-11268), NHDF (normal human dermal fibroblasts) or HT-29 (colonic epithelial tumor cell line, ATCC HTB-38) cells, or a human embryonic kidney-293T (HEK-293T, ATCC CRL-11268), NHDF (normal human dermal fibroblasts) or HT-29 (colonic epithelial tumor cell line, ATCC HTB-38) cell conditioned medium; and selecting cells from the cell population produced by steps a), b) and c) that express one or more of CD4, interleukin (IL)-7 receptor alpha, LTalpha, LTbeta, CD30 ligand, CCR7, CD45, and Integrin β7 protein or mRNA, thereby producing a population of human lymphoid tissue inducer (LTi) cells. Additional selection/screening steps can be included in such methods, for example, selecting cells from the cell population for absence of expression of one or more of IL-17A, IL-22 CD3, CD14, CD16, CD19, CD56, natural killer (NK)-activating receptor NKp44, CD11c, CD11b, human leukocyte antigen DR-1 (HLA-DR), T cell receptor (TCR)-alpha and -beta chains (TCR alpha/beta), recombination activating gene 1 (Rag1), OX40-ligand, LIGHT (lymphotoxin-like, exhibits inducible expression, and competes with HSV glycoprotein D for HVEM, expressed by T lymphocytes), CC chemokine receptor 6 (CCR6), CXC chemokine receptor 5 (CXCR5), B cell marker B220, CD11b, IFN-gamma, inhibitor of DNA binding 2 (Id2) or retinoid-related orphan receptor gamma (Rorc) protein or mRNA expression.

In accordance with the invention, methods of expanding or increasing numbers of LTi cells are provided. Such methods include in vitro or ex vivo contacting LTi cells with a cell or conditioned medium, such as a human embryonic kidney-293T (HEK-293T, ATCC CRL-11268), NHDF (normal human dermal fibroblasts) or HT-29 (colonic epithelial tumor cell line, ATCC HTB-38) cells, or a human embryonic kidney-293T (HEK-293T, ATCC CRL-11268), NHDF (normal human dermal fibroblasts) or HT-29 (colonic epithelial tumor cell line, ATCC HTB-38) cell conditioned medium, under conditions that expand or increase numbers of LTI cells.

In accordance with the invention, methods of identifying a compound that increases or decreases proliferation, growth, survival, or viability of LTi cells are provided. Such methods include, for example, contacting the LTi cells with a test compound, and determining whether the test compound increases or decreases proliferation, growth, survival or viability of LTi cells. A test compound that modulates proliferation, growth, survival or viability of LTi cells can be identified as such.

In accordance with the invention, methods of treating a subject in need of or that would benefit from LTi cells are provided. Such methods include, for example, administering LTi cells to a subject thereby providing the subject with the LTi cells. Candidate subjects treatable by such methods include those with a viral infection, or compromised lymphoid structures, or that are immunocompromised or immunosuppressed.

### DETAILED DESCRIPTION

The invention provides lymphoid tissue inducer (LTi) cells, which have been isolated and/or purified. LTi cells of the invention are characterized by expression of certain markers, such as lineage or developmental markers. In one embodiment, LTi cells are characterized by expression of CD4, interleukin (IL)-7 receptor alpha, LTalpha, LTbeta, CD30 ligand, CC chemokine receptor 7 (CCR7), CD45, and Integrin β7 protein or mRNA.

The invention also provides cultures of lymphoid tissue inducer (LTi) cells that include a growth or storage medium, or are co-cultured with one or more other cell types. In one embodiment, such “cultures” of LTi cells are characterized by expression of CD4, interleukin (IL)-7 receptor alpha, LTalpha, LTbeta, CD30 ligand, CC chemokine receptor 7 (CCR7), CD45, and Integrin β7 protein or mRNA.

Lymphoid tissue inducer (LTi) cells of the invention can optionally be characterized by expression of additional certain markers, such as lineage or developmental markers. In particular aspects, LTi cells are further characterized by expression of inhibitor of DNA binding 2 (Id2) or retinoid-related orphan receptor gamma (Rorc) protein or mRNA.

Lymphoid tissue inducer (LTi) cells of the invention can also optionally be characterized by absence of expression of additional certain markers, such as lineage or developmental markers. In particular aspects, LTi cells are further characterized by absence of expression of one or more of: IL-17A, IL-22 CD3, CD14, CD16, CD19, CD56, natural killer (NK)-activating receptor NKp44, CD11c, CD11b, human leukocyte antigen DR-1 (HLA-DR), T cell receptor (TCR)-alpha and -beta chains (TCR alpha/beta), recombination activating gene 1 (Rag1), OX40-ligand, LIGHT (lymphotoxin-like, exhibits inducible expression, and competes with HSV glycoprotein D for HVEM, expressed by T lymphocytes), CC chemokine receptor 6 (CCR6), CXC chemokine receptor 5 (CXCR5), B cell marker B220, CD11b, or IFN-gamma, protein or mRNA expression.

Lymphoid tissue inducer (LTi) cells of the invention can also optionally be characterized by particular functions or activities, in vitro (e.g., in a culture) or in vivo. In particular aspects, LTi cells are further characterized by one or more of: inducing or stimulating T cells to produce CC chemokine ligand 21 (CCL21), CC chemokine ligand 19 (CCL19), or CXC chemokine ligand 13 (CXCL13), or inducing or stimulating proliferation or differentiation of myeloid dendritic cells (DC). In additional particular aspects, LTi cells are characterized by an ability to migrate to lymph nodes, to Peyer's patches, or to spleen white pulp. In further particular aspects, LTi cells are characterized by contributing to or participating in restoration or formation of lymph nodes, Peyer's patches, or spleen white pulp. In yet additional particular aspects, LTi cells are characterized by stimulating or inducing stromal cells to upregulate an adhesion molecule (e.g., intercellular adhesion molecule 1 (ICAM-1 or CD54) or vascular cell adhesion molecule 1 (VCAM-1 or CD106)), or respond to a cytokine (e.g., IL-7). Such functions or activities occur in vitro (e.g., in a culture) or in vivo.

Lymphoid tissue inducer (LTi) cells of the invention can also optionally be characterized by particular differentiative ability or inability. In particular aspects, LTi cells are further characterized by one or more of: ability to differentiate into natural killer (NK) cells, antigen presenting cells (APC), or dendritic cells (DC), or an inability to differentiate into T cells or B cells.

Lymphoid tissue inducer (LTi) cell cultures include a growth or storage medium (e.g., a cell conditioned medium), or are co-cultured with one or more other cell types. In particular embodiments, a culture of LTi cells includes a human embryonic kidney-293T (HEK-293T, ATCC CRL-11268), NHDF (normal human dermal fibroblasts) or HT-29 (colonic epithelial tumor cell line, ATCC HTB-38) cell conditioned medium. In additional particular embodiments, a culture of LTi cells is a co-culture with human embryonic kidney-293T (HEK-293T, ATCC CRL-11268), NHDF (normal human dermal fibroblasts) or HT-29 (colonic epithelial tumor cell line, ATCC HTB-38) cells.

In LTi cell cultures, in particular aspects, the cells are viably maintained or sustained (survive). In LTi cell cultures, in additional particular aspects, the cells are proliferating or increasing in numbers (i.e., expanding).

LTi cells of the invention are mammalian. Mammalian LTi cells include non-human primate LTi cells (apes, gibbons, gorillas, chimpanzees, orangutans, macaques), human LTi cells, and Lti cells of domestic animals (dogs and cats), farm animals (chickens, ducks, horses, cows, goats, sheep, pigs), and other animals (mouse, rat, rabbit, guinea pig). Lymphoid tissue inducer (LTi) cells are typically adult cells, i.e., that is not an embryo or fetus, such as from a donor (e.g., a mammal such as a human) from about 1 hour old to about 100 years in age.

LTi cells can be genetically or otherwise modified. For example, LTi cells includes cells that do not express a functional protein, such as a signaling protein or express a non-functional protein, such as a signaling protein. LTi cells include those transfected with a nucleic acid encoding an antisense or a functional protein. Non-limiting examples of proteins include ligands, growth factor, cytokines and their corresponding receptors. Such cells are useful for, among other things, methods set forth herein including methods of identifying a compound that or affects (e.g., increases or decreases) proliferation, growth, survival, or viability of LTi cells.

The term “isolated,” when used as a modifier of a composition (e.g., LTi cells, compounds, cytokines, chemokines, growth factors, antibodies, etc.), means that the composition is separated, completely or at least in part, from the naturally occurring in vivo environment. Generally, isolated compositions are substantially free of one or more materials with which they normally associate with in nature, for example, one or more tissues, cells, proteins, or nucleic acid.

An “isolated” composition (e.g., LTi cells) can also be “substantially pure” or “purified” when free of most or all of the materials with which it typically associates with in nature. Thus, isolated LTi cells that also are substantially pure or purified have reduced amounts of contaminants such as other cells or tissues that they normally associated with in vivo, for example. Purified can be at least about 50%, 60% or more by cell numbers or by mass. Purity can also be about 70% or 80% or more, and can be greater, for example, 90% or more. Purity can be less, for example, in a pharmaceutical carrier the amount of LTi cells by weight % can be less than 50% or 60% of the mass by weight, but the relative proportion of the cells compared to other components with which it is normally associated with in nature will be greater. Purity of a population or composition of cells can be assessed by appropriate methods that would be known to the skilled artisan.

A “substantially pure” or “purified” composition (LTi cells) can be combined with one or more other compositions. Thus, “substantially pure” or “purified” does not exclude combinations of compositions, such as combinations of LTi cells co-cultured with other cells (e.g., HEK cells).

LTi cell marker expression is represented by presence of RNA (e.g., mRNA) or protein, or absence of RNA (e.g., mRNA) or protein. Thus, for a given marker, either the RNA encoding the marker or the marker protein is expressed. In certain embodiments, both the marker RNA and marker protein are expressed. Thus, LTi cells can be identified, isolated, purified and/or enriched based upon selecting for a particular pattern of marker RNA and/or marker protein expression, or absence of marker RNA and/or marker protein expression (e.g., removing cells that express a marker that the LTi cells do not), for example. Such marker RNA and/or marker protein expression, or absence of marker RNA and/or marker protein expression can be temporal or transient, such as for a period of 1-10, 20-20, 20-50, 50-100, 100-500 minutes, hours, days, weeks or months.

In accordance with the invention, there are provided methods of producing lymphoid tissue inducer (LTi) cells (e.g., human LTi cells). In one embodiment, a method includes removing or depleting cells expressing CD3 and CD19 from blood cells of a human donor thereby producing a cell population depleted of cells expressing CD3 and CD19; removing or depleting cells expressing CD41 and CD14 from the cell population to produce a cell population depleted of cells expressing CD41 and CD14; contacting the cell population depleted of cells expressing CD3, CD19, CD41 and CD14 with IL-7; co-culturing the cell population with human embryonic kidney-293T (HEK-293T, ATCC CRL-11268), NHDF (normal human dermal fibroblasts) or HT-29 (colonic epithelial tumor cell line, ATCC HTB-38) cells, or a human embryonic kidney-293T (HEK-293T, ATCC CRL-11268), NHDF (normal human dermal fibroblasts) or HT-29 (colonic epithelial tumor cell line, ATCC HTB-38) cell conditioned medium; and selecting cells from the cell population produced that express one or more of CD4, interleukin (IL)-7 receptor alpha, LTalpha, LTbeta, CD30 ligand, CCR7, CD45, and Integrin β7 protein or mRNA, thereby producing a population of human lymphoid tissue inducer (LTi) cells.

In additional aspects, a method further includes selecting cells from the cell population for absence of expression of one or more of markers, such as IL-17A, IL-22 CD3, CD14, CD16, CD19, CD56, natural killer (NK)-activating receptor NKp44, CD11c, CD11b, human leukocyte antigen DR-1 (HLA-DR), T cell receptor (TCR)-alpha and -beta chains (TCR alpha/beta), recombination activating gene 1 (Rag1), OX40-ligand, LIGHT, CC chemokine receptor 6 (CCR6), CXC chemokine receptor 5 (CXCR5), B cell marker B220, CD11b or IFN-gamma, protein or mRNA expression; or selecting cells from the cell population produced for expression of inhibitor of DNA binding 2 (Id2) or retinoid-related orphan receptor gamma (Rorc) protein or mRNA. Such cells can be further purified, isolated or enriched LTi cells by cell sorting, or by selecting for or against functions or activities, or differentiative capability, or other characteristics of LTi cells, as set forth herein.

LTi cells can be viably maintained or survive in growth medium or in co-cultures with other cell types. LTi cells can also undergo proliferation for one or more cell divisions (doublings) so as to increase numbers or expand LTi cells. Thus, in accordance with the invention, there are provided in vitro and ex vivo methods of viably maintaining, as well as proliferating, increasing numbers or expanding LTi cells. In one embodiment, a method includes culturing LTi cells in a growth medium or a co-culture under conditions allowing the cells to proliferate. In one aspect, LTi cells are contacted with human embryonic kidney-293T (HEK-293T, ATCC CRL-11268), NHDF (normal human dermal fibroblasts) or HT-29 (colonic epithelial tumor cell line, ATCC HTB-38) cells, or a human embryonic kidney-293T (HEK-293T, ATCC CRL-11268), NHDF (normal human dermal fibroblasts) or HT-29 (colonic epithelial tumor cell line, ATCC HTB-38) cell conditioned medium under conditions that viably maintain LTI cells, or expand or increase numbers of LTI cells. In particular aspects, LTi cells proliferate or increase in numbers with less than 25%, 20%, 15%, 10%, 5% or less of the cells undergoing cell differentiation. In such methods, proliferating, increasing numbers or expanding LTi cells for a desired number of cell divisions (doublings) thereby produces increased numbers or a population or plurality of LTi cells. Relative proportions or amounts of LTi cells within cell cultures include 1%, 5%, 10%, 20%, 30%, 40%, 50%, 60%, 70%, 80%, 90% or more of the cells in a population or plurality of cells are LTi cells.

A primary isolate of LTi cells can originate from or be derived from a source, such as blood. Progeny of primary isolate LTi cells, which include descendents of the first, second, third and any and all subsequent generations and cells taken or obtained from a primary isolate, that maintain LTi (e.g., LTi cell phenotypic marker expression profile, LTi cell function or activity, etc.) can be obtained from a primary isolate or subsequent expansion of a primary isolate. Subsequent expansion results in progeny LTi cells that can in turn comprise the populations or pluralities of LTi cells, the LTi cell cultures, LTi cell co-cultures, etc. Thus, LTi cells of the invention refer to cells from a primary isolate, for example, from blood, and any progeny cell therefrom. Thus, LTi cells mean that the LTi cells or parental cells of any previous generation at one point in time originated from a source, such as blood. Accordingly, LTi cells are not limited to those from a primary isolate, but can be any subsequent progeny thereof or any subsequent doubling of the progeny thereof provided that the LTi cells express appropriate phenotypic markers, or any other characteristic function, activity or feature set forth herein.

Culture of LTi cells may be carried out in growth medium with or without serum. Serum can range from 0% (v/v) (i.e., serum-free) to about 20% (v/v). Other components, such as growth or survival factors can be included in the growth medium. Growth factors include, for example, FGF-2; basic FGF (bFGF); platelet-derived growth factor (PDGF), epidermal growth factor (EGF), insulin-like growth factor, or insulin. The growth factors may be used alone or in combination. The concentration of growth factor(s) may be from about 4 ng/ml to about 50 ng/ml, from about 10 ng/ml to about 30 ng/ml, from about 5 ng/ml to about 40 ng/ml, from about 10 ng/ml to about 50 ng/ml, from about 50 ng/ml to about 100 ng/ml, or from about 50 ng/ml to about 75 ng/ml.

As used herein, a “cell culture” refers to the maintenance or growth of one or more cells in vitro or ex vivo. Thus, an LTi cell culture is one or more LTi cells in a growth medium. A “culture medium” and a “growth medium” are used interchangeably herein to mean any substance or preparation used for growing, sustaining or maintaining viable cells.

A “co-culture” is a mixed cell population which include LTi cells, and a second cell distinct from the LTi cells (e.g., EK-293T, NHDF or HT-29 cells). A second cell can comprise a population of cells.

Cell cultures and co-cultures of LTi cells can take on a variety of formats. For example, a “suspension culture” refers to a culture in which cells are dispersed throughout a growth medium and can be viable or proliferate while in suspension. An “adherent culture” refers to a culture in which cells in contact with a suitable growth medium are present, and can be viable or proliferate while adhered to a substrate. A “continuous flow culture” refers to the cultivation of cells in a continuous flow of fresh medium to maintain cell viability, e.g. growth.

In a population of LTi cells, or in a culture or co-culture of LTi cells, a majority of cells, but not all cells present may or may not express a particular phenotypic marker indicative of LTi cells. Such cells are typically present in the population, plurality or culture at a smaller percentage of the total number of LTi cells present. In various embodiments, an LTi cell population or a culture of LTi cells include cells in which greater than about 50%, 60%, 70%, 80%, 90%-95% or more (e.g., 96%, 97%, 98%, etc. . . . 100%) of the cells are LTi cells. In various aspects, in a mixed population, such as a co-culture of LTi cells, 50% or less, e.g., 40%, 30%, 25%, 20%, 15%, 10%, 5% or less of the cells may be LTi cells.

A “conditioned media” is a growth medium in which a cell or population of cells are cultured in for a period of time, and then the cells removed. While the cells are cultured in the medium, they secrete factors that include, but are not limited to growth factors, cytokines, extracellular matrix (ECM), proteins, vesicles, antibodies, and granules. The medium with these factors is considered a conditioned medium.

LTi cells, populations and pluralities of LTi cells, LTi cell cultures and co-cultures can be kept or maintained for a period of time (e.g., 1-24 minutes, hours, days, weeks, etc.), can be expanded, or can be allowed to progress to a subsequent developmental, maturation or differentiation stage. LTi cells, populations and pluralities of LTi cells, progeny and methods for expanding, isolating or producing LTi cells, can include growth medium, which can be added or changed at any time, for a period of 1-60 minutes, 1-60 hours or 1-60 days. In exemplary embodiments, fresh growth media is added every 24-48 hours, or during passaging or expanding the cells or following a step of a method of the invention. In additional exemplary embodiments, fresh growth media is added to LTi cells at a given developmental, maturation or differentiation stage, or during cell expansion (proliferation).

LTi cells, including individual clones, populations, pluralities, cultures and co-cultures can be distributed in a vessel or container such as a dish (single or multiwell), plate (single or multiwell), vial, tube, bottle (e.g., roller bottle), flask, bag, syringe or jar. Multi-well dishes and plates include an 8, 16, 32, 64, 96, 384 and 1536 multi-well dish or plate. LTi cells, including individual clones, populations, pluralities, cultures and co-cultures can be attached to a substrate, such as a slide, a dish (single or multiwell), plate (single or multiwell), vial, tube, bottle, or flask.

LTi cells, including individual clones, populations, pluralities, cultures and co-cultures include storing, stored, preserving and preserved forms. In various embodiments, storing, stored, preserving and preserved LTi cells include freezing (frozen) or storing (stored) LTi cells, such as, for example, individual LTi cell clones, a population or plurality of LTi cells, a culture of LTi cells, co-cultures and mixed populations of LTi cells and other cell types. LTi cells and conditioned medium can be preserved or frozen, for example, under a cryogenic condition, such as at −20 degrees C. or less, e.g., −70 degrees C. Preservation or storage under such conditions can include a membrane or cellular protectant, such as dimethylsulfoxide (DMSO).

LTi cells can be used to identify and/or screen for compounds that or affect or modulate (e.g., increase or decrease) viability, survival, proliferation, or growth of LTi cells. For example, a factor(s) secreted or produced by HEK-293T cells appears to contribute to LTi cell viability, survival, proliferation, or growth. Identification of such a factor (e.g., in a HEK-293T cell culture supernatant) The invention therefore provides methods of identifying or screening for a compound that or affects or modulates (e.g., increases or decreases) viability, survival, proliferation, or growth of LTi cells. In one embodiment, a method includes contacting LTi cells with a test compound, and determining whether the test compound modulates or affects (e.g., increases or decreases) proliferation, growth, survival or viability of LTi cells.

Test compounds suitable for analysis include portions or fractions of cell culture supernatants from cells that support LTi cell viability, survival, proliferation, or growth. Thus, test compounds include fractionated (separated) cell culture supernatant based upon size (e.g., size exclusion chromatography), charge (anionic or cationic chromatography) or hydrophobicity (e.g., HPLC chromatography). Exemplary cell culture supernatants that can be fractionated and analyzed according to a method of the invention include HEK-293T, NHDF and HT-29 cell culture supernatants.

Test compounds suitable for analyzing the effect on proliferation also include biologics and pharmacological agents. Such compounds can be peptides, proteins, nucleic acids or organic compounds such as small molecules.

Particular test compounds suitable for analyzing the effect on proliferation include growth factors. Non-limiting examples of growth factors include transforming growth factor-beta (TGF-beta), epidermal growth factor (EGF), platelet derived growth factor (PDGF), fibroblast growth factors (FGFs), such as FGF-1 and FGF-2 (FGF2), transforming growth factors (TGFs) such as TGFalpha and TGFbeta, insulin-like growth factors (IGFs) such as IGF-1 and IGF-2, tumor necrosis factors (TNFs), such as TNFalpha and TNFbeta, interferons (INFs) such as INFgamma, colony stimulating factors (CSFs), such as

Particular test compounds suitable for analyzing the effect on proliferation additionally include cytokines and chemokines. Non-limiting examples of cytokines and chemokines include but are not limited to, interferon (“IFN”)-alpha, IFN-beta, IFN-gamma, tumor necrosis factor (“TNF”)-alpha, Flt3 ligand, interleukin (“IL”)-1 (IL-1alpha, IL-1beta), IL-2, IL-3, IL-4, IL-5, IL-6, IL-7, IL-8, IL-9, IL-10, IL-12, IL-15, IL-18, colony-stimulating factor (“CSF”)-1, granulocyte colony-stimulating factor (“G-CSF”), macrophage colony-stimulating factor (“M-CSF”), granulocyte macrophage colony-stimulating factor (“GM-CSF”), macrophage inflammatory protein (“MIP”)-1, MIP-2, MCP1, gamma interferon inducible protein (“IP-10”) and monokine induced by IFN-gamma (“MIG”). Additional examples include CCL5 (RANTES), CCL6, CCL7 CCL8 (MCP2), CCL9/10, CCL11, CCL12 (MCP5), CCL13, and CCL14-CCL28; CXCL1, CXCL2, CXCL3, CXCL4, CXCL5, CXCL6, CXCL7, CXCL8, CXCL9, and CXCL10-CXCL17; XCL1, XCL2, and CX3CL1.

Particular test compounds suitable for analyzing the effect on proliferation further include ligands and antibodies. Non-limiting examples of ligands and antibodies include ligands and antibodies that bind to growth factors and cytokines, and growth factor receptors and cytokine receptors.

Particular test compounds suitable for analyzing the effect on proliferation moreover include drugs or small molecules. Small molecule test compounds can be organic or inorganic. The small molecules can be natural or non-natural products or members of a combinatorial chemistry library. Drugs and small molecules include, but are not limited to, peptides, peptidomimetics, amino acids, amino acid analogs, polynucleotides, polynucleotide analogs, nucleotides, nucleotide analogs, organic and inorganic compounds (i.e., including hetero-organic and organometallic compounds), and drugs having a molecular weight between about 1-10,000 grams per mole, for example, having a molecular weight between about 10-5,000 grams per mole, having a molecular weight between about 50-1,000 grams per mole, or having a molecular weight between about 100-500 grams/mole. Exemplary small molecules are organic or inorganic molecules having a molecular weight less than about 3,000 Daltons.

Test compounds can be libraries of compounds, which are a plurality of identified or unidentified compounds. Non-limiting examples of libraries include a chemical compound library, or a peptide, protein or antibody library. A number of such libraries are commercially available.

As used herein, the terms “antibody” and “antibodies” refer to proteins that bind to other molecules (antigens) via heavy and light chain variable domains, V_{H} and V_{L}, respectively. Antibodies include full-length antibodies that include two heavy and two light chain sequences. Antibodies can have kappa or lambda light chain sequences, either full length as in naturally occurring antibodies, mixtures thereof (i.e., fusions of kappa and lambda chain sequences), and subsequences/fragments thereof. Naturally occurring antibody molecules contain two kappa or two lambda light chains. Antibodies include multispecific antibodies, human antibodies, humanized antibodies, and chimeric antibodies. Antibodies include monoclonal or polyclonal immunoglobulin molecules that belong to any class such as IgM, IgG, IgA, IgE, IgD, and any subclass thereof. Exemplary subclasses for IgG are IgG₁, IgG₂, IgG₃ and IgG₄. A “monoclonal” antibody refers to an antibody that is based upon, obtained from or derived from a single clone, including any eukaryotic, prokaryotic, or phage clone. A “monoclonal” antibody is therefore defined structurally, and not by the method by which it is produced.

Antibodies include immunoglobulin molecules and immunologically active subsequences or fragments of immunoglobulin molecules, e.g., that contain an antigen binding site. Antibody subsequences and fragments, including single-chain antibodies, can include all or a portion of heavy or light chain variable region sequences (e.g., CDR1, CDR2 or CDR3) alone or in combination with all or a portion of one or more of the following: hinge region, CH1, CH2, and CH3 domains. Non-limiting representative fragments and subsequences of a full length antibody include but are not limited to Fab, Fab′, F(ab′)₂, Fv, Fd, single-chain Fv (scFv), disulfide-linked Fvs (sdFv), V_{L}, V_{H}, trispecific (Fab₃), bispecific (Fab₂), diabody ((V_{L}-V_{H})₂ or (V_{H}-V_{L})₂), triabody (trivalent), tetrabody (tetravalent), minibody ((scF_{V}-C_{H}3)₂), bispecific single-chain Fv (Bis-scFv), IgGdeltaCH2, scFv-Fc, (scFv)₂-Fc and IgG4PE.

Methods of producing polyclonal and monoclonal antibodies are known in the art. For example, an antigen or immunogenic fragment thereof, optionally conjugated to a carrier such as keyhole limpet hemocyanin (KLH) or ovalbumin (e.g., BSA), or mixed with an adjuvant such as Freund's complete or incomplete adjuvant, and used to immunize an animal. Using hybridoma technology, splenocytes from immunized animals that respond to the antigen can be isolated and fused with myeloma cells. Monoclonal antibodies produced by hybridomas can be screened for reactivity. Hybridoma, recombinant, and phage display methods are known in the art (see, for example, U.S. Pat. Nos. 4,902,614, 4,543,439, and 4,411,993; see, also Monoclonal Antibodies, Hybridomas: A New Dimension in Biological Analyses, Plenum Press, Kennett, McKearn, and Bechtol (eds.), 1980, and Harlow et al., Antibodies: A Laboratory Manual, Cold Spring Harbor Laboratory Press, 2nd ed. 1988).

Animals that may be immunized include primates, mice, rats, rabbits, goats, sheep, cattle, or guinea pigs. Initial and any optional subsequent immunization may be through intravenous, intraperitoneal, intramuscular, or subcutaneous routes. Additionally, to increase the immune response, antigen can be coupled to another protein such as ovalbumin or keyhole limpet hemocyanin (KLH), thyroglobulin and tetanus toxoid, or mixed with an adjuvant such as Freund's complete or incomplete adjuvant. Initial and any optional subsequent immunization may be through intraperitoneal, intramuscular, intraocular, or subcutaneous routes. Subsequent immunizations may be at the same or at different concentrations of antigen, and may be at regular or irregular intervals.

Animals include mammals genetically modified to include human gene loci, such as human immunoglobulin lambda or kappa light chain, which can be used to produce human antibodies. Transgenic (e.g., transchromosomic) animals with one or more human immunoglobulin genes are described, for example, in U.S. Pat. No. 5,939,598, WO 02/43478, and WO 02/092812. Human trans-chromosomic mice (KM Mice™) are described, for example, in WO 02/43478, WO 02/092812, and Ishida, et al., IBC's 11^{th} Antibody Engineering Meeting. Abstract (2000)). Such animals include, for example, mice, rat, guinea pig, rabbit, sheep, cow pig and horse.

Humanized antibodies can be produced using techniques known in the art including, for example, CDR-grafting (EP 239,400; WO91/09967; U.S. Pat. Nos. 5,225,539; 5,530,101; and 5,585,089), veneering or resurfacing (EP 592,106; EP 519,596; Padlan, Molecular Immunol. 28:489 (1991); Studnicka et al., Protein Engineering 7:805 (1994); Roguska. et al., Proc. Nat'l. Acad. Sci. USA 91:969 (1994)), and chain shuffling (U.S. Pat. No. 5,565,332). Human consensus sequences (Padlan, Mol. Immunol. 31:169 (1994); and Padlan, Mol. Immunol. 28:489 (1991)) have previously used to produce humanized antibodies (Carter et al., Proc. Natl. Acad. Sci. USA 89:4285 (1992); and Presta et al., J. Immunol. 151:2623 (1993)). Additional methods for producing human polyclonal antibodies and human monoclonal antibodies are described (see, e.g., Kuroiwa et al., Nat. Biotechnol. 20:889 (2002); WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; and 5,939,598).

Methods for producing chimeric antibodies are known in the art (e.g., Morrison, Science 229:1202 (1985); Oi et al., BioTechniques 4:214 (1986); Gillies et al., J. Immunol. Methods 125:191 (1989); and U.S. Pat. Nos. 5,807,715; 4,816,567; and 4,816,397). Chimeric antibodies in which a variable domain from an antibody of one species is substituted for the variable domain of another species are described, for example, in Munro, Nature 312:597 (1984); Neuberger et al., Nature 312:604 (1984); Sharon et al., Nature 309:364 (1984); Morrison et al., Proc. Nat'l. Acad. Sci. USA 81:6851 (1984); Boulianne et al., Nature 312:643 (1984); Capon et al., Nature 337:525 (1989); and Traunecker et al., Nature 339:68 (1989).

LTi cells of the invention have various functions or activities. For example, LTi cells are believed to induce formation or production, or restoration, of lymphoid tissue. In addition, LTi cells of the invention migrate to lymphoid tissues in vivo. For example, LTi cells aggregate or are otherwise located, housed or sequestered in lymphoid tissue. Lymphoid tissue includes secondary lymphoid tissue (e.g., thymus, lymph nodes, spleen white pulp, germinal centers, tonsils, and mucosa-associated lymphoid tissue). Thus, LTi cells of the invention can be used to induce, stimulate or increase formation or production, or restoration, of lymphoid tissue (e.g., thymus, lymph nodes, spleen white pulp, germinal centers, tonsils, and mucosa-associated lymphoid tissue) in vivo or ex vivo. Accordingly, LTi cells and methods of the invention include administering LTi cells to induce, stimulate or increase formation or production, or restoration, of lymphoid tissue, as well as treat pathologies that compromise lymphoid tissue structure, function, formation or integrity in vivo.

In accordance with the invention, there are provided methods of treating a subject in need of or that would benefit from LTi cells. In one embodiment, a method includes administering LTi cells to a subject (e.g., LTi cells autologous to the subject) thereby providing the subject with the LTi cells in an amount sufficient to provide the subject with a benefit. In a particular aspect, a subject has compromised lymphoid structures. In another aspect, a subject is immunocompromised or immunosuppressed. In another aspect, a subject has a chronic or acute viral infection.

The invention treatment methods include, among other things, therapeutic and prophylactic methods. Subjects can be contacted with, administered ex vivo or in vivo delivered Lti cells prior to, concurrently with or following compromised lymphoid structures, or immunosuppression, viral exposure or contact, viral infection, development of a symptom or pathology associated with or caused by compromised lymphoid structures, immunosuppression, or a viral infection or pathogenesis, or reactivation from latency.

The term “therapeutic” and grammatical variations thereof means the subject has the condition, for example, the subject exhibits one or more symptoms or pathologies associated with or caused by compromised lymphoid structures, immunosuppression, a viral infection or pathogenesis, or reactivation from latency as set forth herein or known in the art. The term “therapeutic” also includes a subject that has compromised lymphoid structures, immunosuppression, or a viral infection, but may not exhibit one or more symptoms or pathologies associated with or caused by compromised lymphoid structures, immunosuppression, or a viral infection or pathogenesis.

“Prophylaxis” and grammatical variations thereof refer to contact, administration or in vivo delivery to a subject prior to known development of a condition. In situations where it is not known if a subject has compromised lymphoid structures, immunosuppression, or a viral infection or pathogenesis, contact with, administration or in vivo delivery of LTi cells to a subject occurs prior to manifestation or onset of a symptom associated with or caused by compromised lymphoid structures, immunosuppression, or a viral infection or pathogenesis, or reactivation from latency. In such methods, the effect of contact with, administration or in vivo delivery of LTi cells can be to eliminate, prevent, inhibit, decrease or reduce the probability of or susceptibility towards developing compromised lymphoid structures, immunosuppression, or a viral infection or pathogenesis, or reactivation from latency. Prophylactic methods are applicable to treat subjects at increased risk (probability or susceptibility) of compromised lymphoid structures, immunosuppression, or a viral infection or pathogenesis, or reactivation from latency, for example.

In accordance with the invention, there are provided methods for decreasing or inhibiting viral infection or pathogenesis of a cell in vitro, ex vivo or in vivo, a symptom or pathology associated with a viral infection or pathogenesis in vitro, ex vivo or in vivo, or an adverse side effect of viral infection or pathogenesis in vitro, ex vivo or in vivo. In one embodiment, a method of the invention includes administering LTi cells to a subject, wherein the subject is in need of treatment with an anti-viral activity or function, in order to provide the subject with a beneficial effect or improvement. In another embodiment, a method of the invention includes administering LTi cells to a subject to provide protection against a viral infection or pathogenesis. In a further embodiment, a method of the invention includes administering LTi cells to a subject with a viral infection or pathogenesis to treat the subject for the viral infection or pathogenesis. Methods of the invention include administering LTi cells to a subject prior to, concurrently with, or following contact of the subject with, exposure of the subject to, infection with a virus; and administering LTi cells to a subject prior to, concurrently with, or following development of a symptom or pathology associated with or caused by viral infection or reactivation. In various aspects, LTi cells are administered prior to (prophylaxis), concurrently with or following infection, contact or exposure of the subject to a virus, or reactivation.

Particular non-limiting examples of viruses include poxvirus, herpesvirus, hepatitis virus, immunodeficiency virus, flavivirus, papilloma virus (PV), polyoma virus, rhabdovirus, a myxovirus, an arenavirus, a coronavirus, adenovirus, reovirus, picornavirus, togavirus, bunyavirus, parvovirus and retrovirus. Specific non-limiting examples of herpesviridae (HV) include an alpha-, beta- or gamma-herpesvirus (e.g., herpes simplex virus-1 (HSV-1), herpes simplex virus-2 (HSV-2), varicella zoster virus (VZV/HHV-3), cytomegalovirus (CMV), Epstein-Barr virus (EBV), human herpes virus-6, -7 or -8 (HHV-6, HHV-7, or HHV-8/Kaposi's sarcoma herpesvirus/KSHV)). Specific non-limiting examples of HIV include HIV-1 and HIV-2. HIV-1 includes groups M, N, O and P, and subtypes (or clades), namely, e.g., A, B, C, D, F, G, H, J and K.

Viruses, such as herpesviridae (HV) and HIV are typically found in biological fluids, cells, tissues or organs, in vivo. Accordingly, viruses present in any biological fluid, cell, tissue or organ, are treatable with the invention compounds and methods, locally, regionally or systemically. In particular embodiments, viruses are present in a biological fluid (e.g., mucus, saliva, blood, serum, plasma, cerebrospinal fluid, urine, or placenta); in a tissue or organ comprising a transplant; in an immune cell, tissue or organ, mucosal cell, tissue or organ, neural cell, tissue or organ, or epithelial cell, tissue or organ. In particular aspects, an immune cell is a T cell or a B cell; a mucosal cell or tissue is mouth, buccal cavity, labia, nasopharynx, esophagus, trachea, lung, stomach, small intestine, vagina, rectum, or colon; a neural cell or tissue is ganglia, motor or sensory neuron; and an epithelial cell or tissue is nose, fingers, ears, cornea, conjunctiva, skin or dermis.

As used herein, the term “associated with,” when used in reference to the relationship between a symptom, pathology or adverse side effect of a condition, such as compromised lymphoid structures, immunosuppression, or a viral infection or pathogenesis, or reactivation from latency, means that the symptom, pathology or side effect is caused by the condition, or is a secondary effect of the condition. A symptom, pathology or side effect that is present in a subject may therefore be the direct result of or caused by the condition (e.g., compromised lymphoid structures, immunosuppression, or a viral infection or pathogenesis, or reactivation from latency), or may be due at least in part to the subject reacting or responding to the condition (e.g., the immunological response). For example, a symptom or pathology that occurs during compromised lymphoid structures, immunosuppression, or a viral infection or pathogenesis, or reactivation from latency, may be due in part to an inflammatory or other response of the subject.

Exemplary symptoms of herpesviridae infection include lesions, ulcers, canker sore, cold sore, rash, boils, Gingivostomatitis, Herpetic whitlow Traumatic herpes (herpes gladiatorum), Eczema herpeticum, fever, fatigue, headache, sore throat, swollen lymph nodes, pneumonitis, pneumonia, hepatitis, meningitis, myelitis, Encephalitis, keratitis, Genital herpes, esophagitis, dysphasia, hemiparesis, coma, shingles, chicken pox, mononucleosis, chronic or acute pelvic inflammatory disease (PID), proctitis, colitis, nerve damage, death, etc.). Exemplary symptoms of HIV infection include reduced numbers of CD4+ T cells, as compared to an age, gender, race, etc. matched subject. For example, a CD4+ T cell count less than 500 cells/microliter blood, or less than 200 cells/microliter blood, or the percentage of CD4+ T cells in the subject is less than 15% of all lymphocytes. Exemplary symptoms of HIV infection also include fever, fatigue, headache, sore throat, swollen lymph nodes, weight loss, diarrhea, rash, boils, warts, thrush, shingles, chronic or acute pelvic inflammatory disease (PID), dry cough, shortness of breath, bruising, bleeding, numbness or paralysis, muscle weakness, an opportunistic disorder, nerve damage, encephalopathy, dementia, death, etc.

Methods of the invention include methods of treatment that results in a beneficial effect. Particular non-limiting examples of beneficial effects include inducing, stimulating or increasing formation or production, or restoration, of lymphoid tissue, as well as treat pathologies that compromise lymphoid tissue structure, function, formation or integrity in vivo; providing a subject with partial or complete protection against virus infection, reactivation or pathogenesis, or a symptom caused by a virus infection, reactivation or pathogenesis (e.g., inhibit or reduce probability or susceptibility). Particular non-limiting examples of beneficial effects also include reducing, decreasing, inhibiting, delaying or preventing virus infection, reactivation or pathogenesis, and reducing, decreasing, inhibiting, ameliorating or preventing onset, severity, duration, progression, frequency or probability of one or more symptoms or pathologies associated with a virus infection, reactivation or pathogenesis. Additional non-limiting examples of beneficial effects also include reducing, decreasing, amounts of, or inhibiting, delaying or preventing increases in viral titer or viral load, proliferation or replication. Further non-limiting particular examples of beneficial effects include reducing, decreasing, inhibiting, delaying, ameliorating or preventing onset, progression, severity, duration, frequency, probability or susceptibility of a subject to compromised lymphoid structures, immunosuppression, or a viral infection or pathogenesis, or accelerating, facilitating or hastening recovery of a subject from compromised lymphoid structures, immunosuppression, a viral infection or pathogenesis, or one or more associated symptoms or pathologies.

The term “infection,” means a initial or primary or a chronic infection. An infection may be “infectious” in the sense that other sites in the infected host subject, or contagious to other subjects (cross-infection), or may be latent. An initial/primary infection can cause mild, moderate or severe pathogenesis or symptoms, or be asymptomatic. A primary/initial infection can be self-limiting, and can progress to latency. A “latent” infection in the host subject is a state in which the infection (e.g., virus) evades immune clearance and remains in the host subject, which infection can be chronic, even lifelong. In the latent state illness or symptoms may not be present or may be mild.

The term “reactivation,” when used in reference to HV, means activation of HV in the host subject following a period of latency. Reactivation is associated with increased viral replication and proliferation in an HIV infected host subject, who becomes infectious and contagious again. Symptoms and pathologies associated with or caused by HV reactivation may or may not be the same type, severity, frequency or duration as initial HIV infection and subsequent pathogenesis. For example, VZV/HHV-3 causes chickenpox (primary infection) and shingles (reactivation). Reactivation can be milder (e.g., asymptomatic) than an initial HV infection/pathogenesis, in which case it would not be obvious whether a host subject is in a latent or reactivated state. In immunocompetent host subjects reactivation is typically mild, whereas in immunocompromised host subjects, symptoms associated with or caused by reactivation can be severe and lead to death. Thus, clinical manifestations associated with reactivation may be different from that observed with an initial/primary infection. One symptom of HV reactivation is the appearance of “cold sores” around mucosal areas (e.g., mouth, lips, tongue, genitalia, etc.). Reactivation occurs periodically and can be induced by stress, immune suppression, etc.

As used herein, a “sufficient amount” or “effective amount” or an “amount sufficient” or an “amount effective” refers to an amount that provides, in single or multiple doses, alone or in combination with one or more other compounds, treatments, agents (e.g., a drug) or therapeutic regimens, a long term or a short term detectable or measurable improvement or beneficial effect to a given subject of any degree or for any time period or duration (e.g., for minutes, hours, days, months, years, or cured).

A “sufficient amount” or “effective amount” therefore includes decreasing, reducing, inhibiting, preventing, or delaying onset; decreasing, reducing, inhibiting, delaying, or preventing a progression or worsening of or reducing, relieving, ameliorating, or alleviating, severity, frequency, duration, susceptibility or probability of one or more symptoms caused by or associated with compromised lymphoid structures, immunosuppression, or a viral infection or pathogenesis, or reactivation, one or more symptoms associated with or caused by compromised lymphoid structures, immunosuppression, or a viral infection or pathogenesis, or reactivation from latency. In addition, hastening a subject's recovery from compromised lymphoid structures, immunosuppression, or a viral infection or pathogenesis, or reactivation from latency, is considered to be a sufficient or effective amount.

A sufficient amount or an effective amount can but need not be provided in a single administration and can but need not be administered alone (i.e., without a second drug, agent, treatment or therapeutic regimen), or in combination with another compound, agent, treatment or therapeutic regimen. In addition, a sufficient amount or an effective amount need not be sufficient or effective if given in single or multiple doses without a second compound, treatment, agent, or therapeutic regimen, since additional doses, amounts, frequency or duration of administration above and beyond such doses, or additional compounds, agents, treatments or therapeutic regimens may be included in order to be effective or sufficient in a given subject.

A sufficient amount or an effective amount need not be effective in each and every subject, nor a majority of subjects in a given group or population. Thus, a sufficient amount or an effective amount means sufficiency or effectiveness in a particular subject, not a group or the general population. As is typical for such methods, some subjects will exhibit a greater or less response to a method of the invention than other subjects.

Amounts, frequencies or duration also considered sufficient and effective and are therefore beneficial are those that result in the elimination or a reduction in amount, frequency or duration of another compound, agent, treatment or therapeutic regimen. For example LTI cells is considered as having a beneficial or therapeutic effect if contact, administration or delivery in vivo results in the use of a lesser amount, frequency or duration of another compound, agent, treatment or therapeutic regimen to treat the condition, such as compromised lymphoid structures, immunosuppression, or a viral infection or pathogenesis, or reactivation from latency, infection, pathogenesis, symptom or pathology.

The terms “subject” and “patient” refer to an animal, typically mammalian animals, such as but not limited to non-human primates (apes, gibbons, gorillas, chimpanzees, orangutans, macaques), domestic animals (dogs and cats), a farm animals (chickens, ducks, horses, cows, goats, sheep, pigs), experimental animal (mouse, rat, rabbit, guinea pig) and humans. Subjects include animal models, for example, a mouse model. Subjects include naturally occurring or non-naturally occurring mutated or non-human genetically engineered (e.g., transgenic or knockout) animals. Human subjects include children, for example, newborns, infants, toddlers and teens, between the ages of 1 and 5, 5 and 10 and 10 and 18 years, adults between the ages of 18 and 60 years, and the elderly, for example, between the ages of 60 and 65, 65 and 70 and 70 and 100 years.

Subjects can be any age. For example, a subject (e.g., human) can be a newborn, infant, toddler, child, teenager, or adult, e.g., 50 years or older.

Subjects include those in need of LTi cells or a method of the invention, e.g., a subject in need of a treatment, therapeutic or prophylactic, with LTi cells, or that would potentially benefit from LTi cells or a method herein. A subject is considered to be in need if LTi cells or a method is likely to provide a benefit to a subject. Subjects appropriate for treatment therefore include, for example, those having or at risk of compromised lymphoid structures, immunosuppression, or a viral infection or pathogenesis, or reactivation from latency. Such subjects include immunocompromised or immunosuppressed subjects due to an immunological disorder (e.g., autoimmunity) or disease, or an immune-suppressing treatment (e.g., cyclophosphamide), subjects that have been exposed to or diagnosed as HIV+; and subjects receiving or candidates for a tissue or organ transplant. A subject may be symptomatic or asymptomatic for compromised lymphoid structures, immunosuppression, or a viral infection or pathogenesis, or reactivation from latency.

Candidate subjects include subjects that have been exposed to or contacted with a virus, or that are at risk of exposure to or contact with a virus, regardless of the type, timing or extent of exposure or contact. The invention methods are therefore applicable to a subject who is at risk of viral infection, reactivation or pathogenesis, but has not yet been exposed to or contacted with virus.

LTi cells can be incorporated into pharmaceutical compositions or formulations. Such pharmaceutical compositions/formulations are useful for administration to a subject, in vivo or ex vivo. Pharmaceutical compositions and formulations include carriers or excipients for administration to a subject. As used herein the terms “pharmaceutically acceptable” and “physiologically acceptable” mean a biologically compatible formulation, gaseous, liquid or solid, or mixture thereof, which is suitable for one or more routes of administration, in vivo delivery or contact. A formulation is compatible in that it does not destroy activity of LTi cells, or induce adverse side effects that far outweigh any prophylactic or therapeutic effect or benefit.

Pharmaceutical compositions can optionally be formulated to be compatible with a particular route of administration. Exemplary routes of administration include administration to a blood vessel, biological fluid, organ or tissue, or lymphoid cell or tissue. Thus, pharmaceutical compositions include carriers (excipients, diluents, vehicles or filling agents) suitable for administration by various routes and delivery, locally, regionally or systemically.

Exemplary routes of administration for contact or in vivo delivery which LTi cells can optionally be formulated include parenteral (e.g., subcutaneous, intramuscular, intravenous, intradermal, intraocular, intratracheal and epidural), intrathecal, intraarticular, intracavity, intraglandular, intraorgan, or intralymphatic.

Formulations suitable for parenteral administration include aqueous and non-aqueous solutions, suspensions, which may include suspending agents and thickening agents, which preparations are typically sterile and can be isotonic with the blood of the intended recipient. Non-limiting illustrative examples of aqueous carriers include water. The formulations may be presented in unit-dose or multi-dose kits, for example, ampules and vials, prior to use.

Pharmaceutical formulations and delivery systems appropriate for the compositions and methods of the invention are known in the art (see, e.g., Remington: The Science and Practice of Pharmacy (2003) 20^{th} ed., Mack Publishing Co., Easton, Pa.; Remington's Pharmaceutical Sciences (1990) 18^{th} ed., Mack Publishing Co., Easton, Pa.; The Merck Index (1996) 12^{th} ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11^{th} ed., Lippincott Williams & Wilkins, Baltimore, Md.; and Poznansky et al., Drug Delivery Systems (1980), R. L. Juliano, ed., Oxford, N.Y., pp. 253-315).

LTi cells, including pharmaceutical formulations can be packaged in unit dosage forms for ease of administration and uniformity of dosage. A “unit dosage form” as used herein refers to a physically discrete unit suited as unitary dosages for the subject to be treated; each unit containing a predetermined quantity of compound optionally in association with a pharmaceutical carrier (excipient, diluent, vehicle or filling agent) which, when administered in one or more doses, is calculated to produce a desired effect (e.g., prophylactic or therapeutic effect or benefit). Unit dosage forms can contain a daily dose or unit, daily sub-dose, or an appropriate fraction thereof, of an administered compound (e.g., LTi cells). Unit dosage forms also include, for example, ampules and vials with liquid compositions disposed therein. The individual unit dosage forms can be included in multi-dose kits or containers. Pharmaceutical formulations can be packaged in single or multiple unit dosage forms for ease of administration and uniformity of dosage.

LTi cells of the invention can be administered at any frequency as a single bolus or multiple dose e.g., one, two, three, four, five, or more times hourly, daily, weekly, monthly or annually or between about 1 to 10 days, weeks, months, or for as long as appropriate. Exemplary frequencies are typically from 1-7 times, 1-5 times, 1-3 times, 2-times or once, daily, weekly or monthly. Timing of contact, administration ex vivo or in vivo delivery can be dictated by the condition to be treated.

Doses may vary depending upon whether the treatment is therapeutic or prophylactic, the onset, progression, severity, frequency, duration, probability of or susceptibility of the symptom, the type of virus infection, reactivation or pathogenesis to which treatment is directed, clinical endpoint desired, previous, simultaneous or subsequent treatments, general health, age, gender or race of the subject, bioavailability, potential adverse systemic, regional or local side effects, the presence of other disorders or diseases in the subject, and other factors that will be appreciated by the skilled artisan (e.g., medical or familial history). Dose amount, frequency or duration may be increased or reduced, as indicated by the clinical outcome desired, status of the infection, reactivation, pathology or symptom, or any adverse side effects of the treatment or therapy. The skilled artisan will appreciate the factors that may influence the dosage, frequency and timing required to provide an amount sufficient or effective for providing a prophylactic or therapeutic effect or benefit.

Doses can be based upon current existing treatment protocols, empirically determined, determined using animal disease models or optionally in human clinical studies. The dose can be adjusted according to the mass of a subject. A subject may be administered in single bolus or in divided/metered doses, which can be adjusted to be more or less according to the various consideration set forth herein and known to one of skill in the art.

Dose amount, frequency or duration may be increased or reduced, as indicated by the status of the condition. For example, once control or a particular endpoint is achieved, for example, reducing, decreasing, inhibiting, ameliorating or preventing onset, severity, duration, progression, frequency or probability of one or more symptoms associated with compromised lymphoid structures, immunosuppression, or a viral infection or pathogenesis, or reactivation from latency, dose amount, frequency or duration can be reduced.

The invention provides kits including LTi cells, combinations thereof and pharmaceutical compositions/formulations thereof, packaged into a suitable packaging material. In one embodiment, a kit includes packaging material, LTi cells and instructions. In various aspects, the instructions are for administering LTi cells to treat compromised lymphoid structures, immunosuppression, or a viral infection or pathogenesis, or reactivation from latency.

The term “packaging material” refers to a physical structure housing one or more components of the kit. The packaging material can maintain the components sterilely, and can be made of material commonly used for such purposes (e.g., paper, corrugated fiber, glass, plastic, foil, ampules, vials, tubes, etc.).

A kit optionally includes a label or insert including a description of the components (type, amounts, doses, etc.), instructions for use in vitro, in vivo, or ex vivo, and any other components therein. Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a disk, optical disk such as CD- or DVD-ROM/RAM, DVD, MP3, magnetic tape, or an electrical storage media such as RAM and ROM or hybrids of these such as magnetic/optical storage media, FLASH media or memory type cards.

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer, lot numbers, manufacturer location and date, expiration dates.

Labels or inserts can include information on a condition, (e.g., compromised lymphoid structures, immunosuppression, or a viral infection or pathogenesis, or reactivation from latency) for which a kit component may be used. Labels or inserts can include instructions for a clinician or subject for using one or more of the kit components in a method, treatment protocol or therapeutic/prophylactic regimen, including the methods of the invention. Instructions can include amounts of compound, frequency or duration of administration, and instructions for practicing any of the methods, treatment protocols or prophylactic or therapeutic regimes described herein. Kits of the invention therefore can additionally include labels or instructions for practicing any of the methods of the invention described herein including treatment or other methods (e.g., identifying/screening).

Labels or inserts can include information on any effect or benefit a kit component may provide, such as a prophylactic or therapeutic effect or benefit. For example, a label or insert could provide a description of one or more symptoms which can be improved, such as inducing, stimulating or increasing formation or production, or restoration, of lymphoid tissue structure, function, formation or integrity in vivo; partial or complete protection against virus infection, reactivation or pathogenesis, or a symptom caused by a virus infection, reactivation or pathogenesis.

Labels or inserts can include information on potential adverse side effects of treatment. Labels or inserts can further include warnings to the clinician or subject regarding situations or conditions where a subject should stop or reduce use of a particular kit component. Adverse side effects could also occur when the subject has, will be or is currently taking one or more other medications that may be incompatible with a compound of the invention, or the subject has, will be or is currently undergoing another treatment protocol or therapeutic regimen which would be incompatible with the compound and, therefore, labels or inserts could include information regarding such side effects or incompatibilities.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All applications, publications, patents and other references, GenBank citations and ATCC citations cited herein are incorporated by reference in their entirety. In case of conflict, the specification, including definitions, will control.

As used herein, the singular forms “a,” “and,” and “the” include plural referents unless the context clearly indicates otherwise. Thus, for example, reference to an “LTi cell” includes a plurality of LTi cells, and reference to an “activity or function” such as “LTi cell activity or function” can include reference to one or more LTi cell activities or functions.

As used herein, all numerical values or ranges include fractions of the values and integers within such ranges and fractions of the integers within such ranges unless the context clearly indicates otherwise. Thus, to illustrate, reference to a numerical range, such as a percentage range, 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. Reference to a range of 1-5 fold therefore includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and so forth. Reference to a series of ranges includes combinations of the upper and lower end of the ranges. For example, reference to a series of ranges from 5-20, 20-50, 50-100, 100-500, includes ranges from 5-50, 5-100, 5-500, 20-100, 20-500, 50-500, etc.

The invention is generally disclosed herein using affirmative language to describe the numerous embodiments and aspects. The invention also specifically includes embodiments in which particular subject matter is excluded, in full or in part, such as substances or materials, method steps and conditions, protocols, procedures, assays or analysis. For example, in certain embodiments or aspects of the invention, other materials and method steps are excluded. Thus, even though the invention is generally not expressed herein in terms of what is not included, embodiments and aspects that expressly exclude compositions or method steps are nevertheless disclosed and included in the invention.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EXAMPLES

### Example 1

### This Example Includes a Description of the Isolation of Human CD4 LTi Cells.

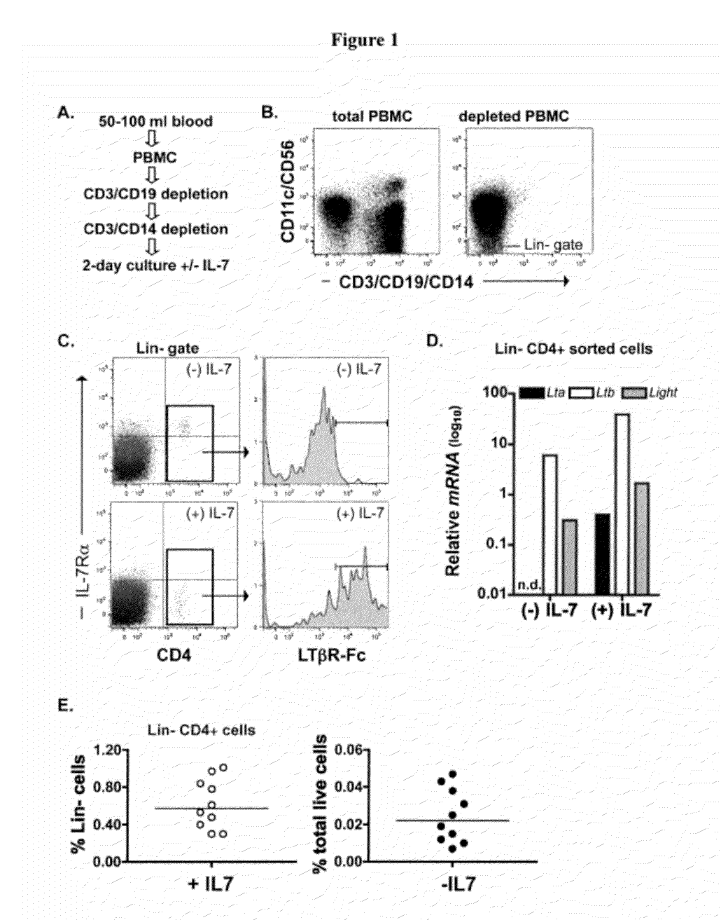

Human LTi cells were isolated as in adult mouse splenicLTi cells (FIG. 1A). Mononuclear cells in adult human blood were isolated and depleted of cells bearing lineage markers for T cells (CD3ε), B lymphocytes (CD19), monocytes (CD14), dendritic cells (CD11c) and NK cells (CD56). Lineage negative (Lin^{neg}) cells were further identified by expression of CD4^{bright} and IL-7Rα, and then purified by high speed cell sorting (FIG. 1B).

Mouse embryonic LTi cells are highly responsive to IL-7 as measured by induction of surface LTαβ (Yoshida, et al., Immunity (2002) 17:823). Human Lin^{neg} cells coexpressing IL-7Rα and CD4 were cultured in IL-7 to assess their expression of Lymphotoxin-LT-related cytokines. LTβ and LIGHT were detected in unstimulated Lin^{neg} cells. Dramatically, IL-7 induced a 10-50 fold increase in binding of the LTβR-Fc to these cells as detected by flow cytometry (FIG. 1C). mRNA for LTα and LTβ increased proportionally with IL-7 treatment, whereas LIGHT mRNA increased 3-4 fold. Together the result implicates the LTαβ complex as the more abundant ligand on the surface of human LTi (FIG. 1D).

The Lin^{neg} cells expressing CD4 represent less than 0.5% of human peripheral blood cells (FIG. 1E). The re-expression of various lineage makers in the Lin^{neg} cell population was determined after culture to determine the extent of contamination by conventional lymphoid cells. Less than 1.0% of Lin^{neg} cells regained expression CD3 after culturing in medium for 48 hrs.

### Example 2

### This Example Includes a Description of the Expansion and Maintenance of Human and Mouse LTi Cells in Tissue Culture.

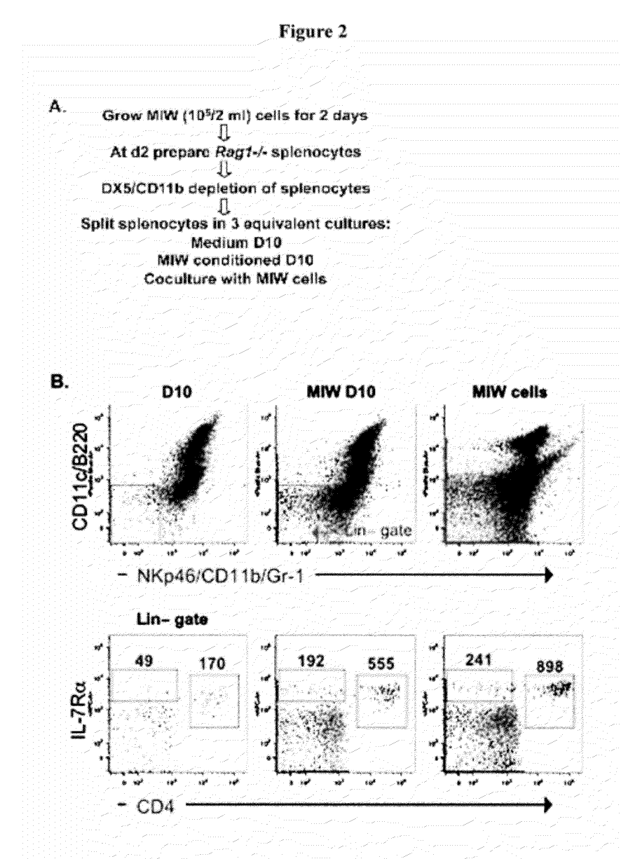

LTi cells were co-cultured with a human embryonic epithelial line, HEK-293T, which created a neogeneic environment that appeared to support survival and expansion of CD4+ Lin^{neg} cells (FIG. 2).

Mouse LTi cells were isolated from splenocytes harvested from RAG mice. RAG^{-/-} mice are genetically deficient in lymphocyte lineages, and have a higher number of LTi cells in their spleen. RAG^{-/-} splenocytes were depleted of NK (DX5) and macrophages (CD11b). The Lin^{neg} cells were cultured in standard culture medium or in medium with HEK-293T cells. Culturing Lin^{neg} cells with embryonic HEK-293T cells promoted a ˜5 fold increase in CD4+ IL-7Rα+ cells (from 177 to 898 cells) relative to medium control in two days of culture. Cells cultured in conditioned medium from HEK-293T cells also promoted LTi expansion, but to a lesser degree. This result suggested a soluble factor secreted from HEK-293T cells may supplant the cell line.

### Example 3

This Example Includes a Description of HEK-293T Cells Providing a Distinct Signal from IL-7 to Lin^{neg} Cells.

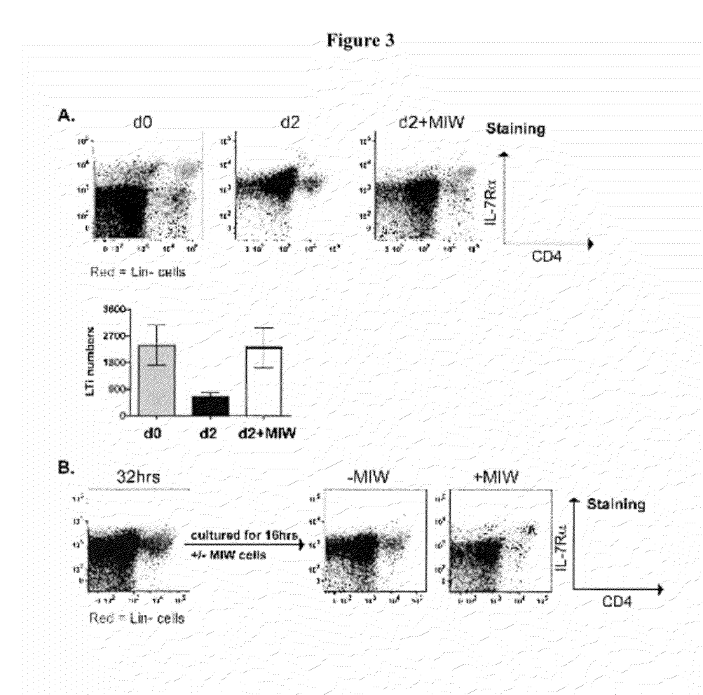

In the RAG−/− spleen, LTi cells are readily distinguishable by their high expression of CD4 and IL-7Rα (FIG. 3A). After 2 days in culture, CD4+Lin^{neg} cells downregulated IL-7Rα (red dots in FIG. 3A); however, when co-cultured with HEK-293T cells, the Lin^{neg} cells re-expressed high IL-7Rα and CD4 (FIG. 3A) characteristic of the LTi phenotype. Cell number increased or was maintained in these cultures over two days indicating the HEK-293T provides minimally cell survival signals for the LTi cells (FIG. 3A bar graph). Furthermore, a short 16 hr co-culture with HEK-293T is sufficient to upregulate IL-7Rα on CD4+Lin^{neg} cells (FIG. 3B). Thus, IL-7 may not be the HEK-293T secreted trophic factor because IL-7 down regulates IL-7Rα in the Lin^{neg} cells (FIG. 1). This observation suggests the HEK-293T cells provide Lin^{neg} cells differentiative signals distinct from IL-7.

### Example 4

This Example Includes Studies Indicating that HEK-293T Cells do not Induce the Differentiation of Putative LTi Progenitors in the Spleen.

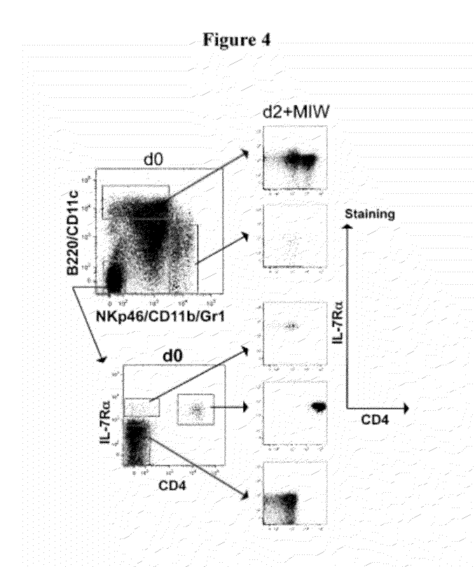

It is plausible that upregulation of IL-7Rα was not due to an intrinsic LTi biological function but due to the emergence of LTi cells through stem cell progenitors residing in the spleen. To analyze this possibility various cell populations from RAG−/− spleens were sorted and co-cultured them with HEK-293T cells. After culture for 2 days the expression of CD4 and IL-7Rα in lineage positive and negative cell populations were measured (FIG. 4). Cells with high level expression of CD4+IL-7Rα+ in the lineage positive cell populations were not detected. Thus, the effects of HEK-293T cells appear to directly impact putative LTi cells

### Example 5

### Re-Evaluation of Putative Human CD4+Lin− LTi Cells Utilizing HEK-293T Co-Cultures.

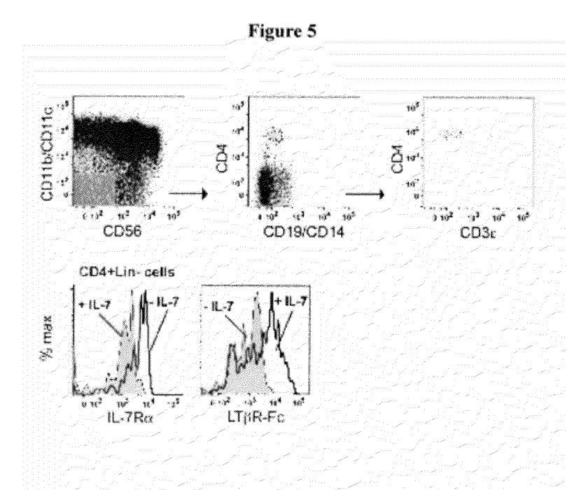

Although CD4+Lin−IL-7Rα+ populations in peripheral human blood were consistently present, cell recovery was very low, typically reaching ˜400 cells after sorting 50-100 ml blood. Based on the results with mouse LTi cultures, the putative human LTi population might survive when co-cultured with HEK-293T cells. Moreover, the flow cytometry staining protocol was improved by including additional dyes, and reducing the number of antibodies in “damp” gates. Following depletion of CD3, CD19, CD14 and CD41 (contaminating platelets) expressing cells, the remaining cells were co-cultured with HEK-293T in the presence or absence IL-7 and stained the cells 2 days later (FIG. 5). Consistent with previous findings a CD4+Lin^{neg} population that expressed IL-7Rα, which was downregulated after IL-7 stimulation was identified. IL-7 induced surface LTβR ligands (FIG. 5). Importantly, the HEK-293T co-culture system improved cell recovery 5-fold, suggesting the potential for cell proliferation.

### Example 6

### This Example Includes a Description of Gene Expression in Human CD4+ Lin^{neg} Cells.

Mouse CD4+IL-7RαLin^{neg}LTi cells express a subset of cytokines, chemokines and transcription factors that provide a distinguishing profile from dendritic cells, plasmacytoid dendritic cells, T cells, B cells, and NK cells (Kim, et al., J Immunol (2006) 177:3074). For example, adult mouse LTi cells only express OX40L and CD30L.

Several genes related to the differentiation of mouse LTi were expressed in human CD4+ Lin^{neg} cells cultured with HEK-293T cells or HEK-293T cells with IL-7 as detected by qRT-PCR (FIG. 6). Human LTi cells expressed high basal mRNA levels of Tnfsf8 (CD30 ligand), Light, Il22, chemokine receptor Cxcr4, and lesser relative level of Il13. IL-7 slightly down modulated mRNA for CD30L and LIGHT, but did not impact the other genes. Rorc, the retinoid orphan nuclear receptor required for differentiation of IL-17 producing Th cells and LTi cells, was upregulated ˜10 fold by IL-7. Basal levels of both Ltβ and Il17A were very low, but were dramatically upregulated (50-100 fold) in LTi cells by IL-7. This result indicates that the HEK-293T culture does not induce differentiation of Lin^{neg} cells in the same way as IL-7, at least as determined by LTαβ and IL-17A expression. The expression of IL-17 by adult blood LTi is consistent with expression profile in the one report of human LTi isolated from fetal tissue (Cupedo, et al., Nat Immunol (2009) 10:66). However, the fetal human LTi like cells isolated in the study by Spits and colleagues, did not express CD4, and appear to give rise to NK cells, suggesting progenitor like cells. The CD4+ IL-7RαLin^{neg} cells isolated from human blood may be derived from a common progenitor, but differentiated in blood or lymphoid tissues.

What is claimed is:

1. Isolated or purified human lymphoid tissue inducer (LTi) cells, wherein the LTi cells are characterized by expression of CD4, interleukin (IL)-7 receptor alpha, LTalpha, LTbeta, CD30 ligand, CC chemokine receptor 7 (CCR7), CD45, and Integrin β7 protein or mRNA.

2. A culture of human lymphoid tissue inducer (LTi) cells comprising a growth or storage medium, wherein the LTi cells are characterized by expression of CD4, interleukin (IL)-7 receptor alpha, LTalpha, LTbeta, CD30 ligand, CCR7, CD45, and Integrin β7 protein or mRNA.

3. The lymphoid tissue inducer (LTi) cells of claim 1 or 2, wherein the LTi cells are further characterized by expression of inhibitor of DNA binding 2 (Id2) or retinoid-related orphan receptor gamma (Rorc) protein or mRNA.

4. The lymphoid tissue inducer (LTi) cells of claim 1 or 2, wherein the LTi cells are further characterized by absence of one or more of: IL-17A, IL-22 CD3, CD14, CD16, CD19, CD56, natural killer (NK)-activating receptor NKp44, CD11c, CD11b, human leukocyte antigen DR-1 (HLA-DR), T cell receptor (TCR)-alpha and -beta chains (TCR alpha/beta), recombination activating gene 1 (Rag1), OX40-ligand, LIGHT (lymphotoxin-like, exhibits inducible expression, and competes with HSV glycoprotein D for HVEM, expressed by T lymphocytes), CC chemokine receptor 6 (CCR6), CXC chemokine receptor 5 (CXCR5), B cell marker B220, CD11b or IFN-gamma, protein or mRNA expression.

5. The lymphoid tissue inducer (LTi) cells of claim 1 or 2, wherein the LTi cells are further characterized by one or more of: inducing or stimulating T cells to produce CC chemokine ligand 21 (CCL21), CC chemokine ligand 19 (CCL19), or CXC chemokine ligand 13 (CXCL13), or inducing or stimulating proliferation or differentiation of myeloid dendritic cells (DC).

6. The lymphoid tissue inducer (LTi) cells of claim 1 or 2, wherein the LTi cells are further characterized by an ability to migrate to lymph nodes, Peyer's patches, or spleen white pulp.

7. The lymphoid tissue inducer (LTi) cells of claim 1 or 2, wherein the LTi cells are further characterized by contributing to or participating in restoration or formation of lymph nodes, Peyer's patches, or spleen white pulp.

8. The lymphoid tissue inducer (LTi) cells of claim 1 or 2, wherein the LTi cells are further characterized by stimulating or inducing stromal cells to upregulate an adhesion molecule.

9. The lymphoid tissue inducer (LTi) cells of claim 8, wherein the adhesion molecule is intercellular adhesion molecule 1 (ICAM-1 or CD54) or vascular cell adhesion molecule 1 (VCAM-1 or CD106).

10. The lymphoid tissue inducer (LTi) cells of claim 1 or 2, wherein the LTi cells respond to IL-7.

11. The lymphoid tissue inducer (LTi) cells of claim 1 or 2, wherein the LTi cells are further characterized by one or more of: ability to differentiate into natural killer (NK) cells, antigen presenting cells (APC), or dendritic cells (DC), and an inability to differentiate into T cells or B cells.

12. The lymphoid tissue inducer (LTi) cells of claim 1 or 2, wherein the cells are adult cells.

13. The lymphoid tissue inducer (LTi) cells of claim 2, wherein the cells are co-cultured with human embryonic kidney-293T (HEK-293T, ATCC CRL-11268), NHDF (normal human dermal fibroblasts) or HT-29 (colonic epithelial tumor cell line, ATCC HTB-38) cells, or human embryonic kidney-293T (HEK-293T, ATCC CRL-11268), NHDF (normal human dermal fibroblasts) or HT-29 (colonic epithelial tumor cell line, ATCC HTB-38) cell conditioned medium.

14. The lymphoid tissue inducer (LTi) cells of claim 1 or 2, wherein the cells are viably maintained or sustained, or are proliferating or increasing in numbers.

15. A method of producing human lymphoid tissue inducer (LTi) cells, comprising
a) removing or depleting cells expressing CD3 and CD19 from blood cells of a human donor thereby producing a cell population depleted of cells expressing CD3 and CD19;
b) removing or depleting cells expressing CD41 and CD14 from the cell population thereby producing a cell population depleted of cells expressing CD41 and CD14;
c) contacting the cell population depleted of cells expressing CD3, CD19, CD41 and CD14 with IL-7;
d) co-culturing the cell population with human embryonic kidney-293T (HEK-293T, ATCC CRL-11268), NHDF (normal human dermal fibroblasts) or HT-29 (colonic epithelial tumor cell line, ATCC HTB-38) cells, or a human embryonic kidney-293T (HEK-293T, ATCC CRL-11268), NHDF (normal human dermal fibroblasts) or HT-29 (colonic epithelial tumor cell line, ATCC HTB-38) cell conditioned medium; and
e) selecting cells from the cell population produced by steps a), b) and c) that express one or more of CD4, interleukin (IL)-7 receptor alpha, LTalpha, LTbeta, CD30 ligand, CCR7, CD45, and Integrin β7 protein or mRNA, thereby producing a population of human lymphoid tissue inducer (LTi) cells.

16. The method of claim 15, comprising selecting cells from the cell population produced by steps a), b) or c) for absence of expression of one or more of IL-17A, IL-22 CD3, CD14, CD16, CD19, CD56, natural killer (NK)-activating receptor NKp44, CD11c, CD11b, human leukocyte antigen DR-1 (HLA-DR), T cell receptor (TCR)-alpha and -beta chains (TCR alpha/beta), recombination activating gene 1 (Rag1), OX40-ligand, LIGHT (lymphotoxin-like, exhibits inducible expression, and competes with HSV glycoprotein D for HVEM, expressed by T lymphocytes), CC chemokine receptor 6 (CCR6), CXC chemokine receptor 5 (CXCR5), B cell marker B220, CD11b or IFN-gamma, protein or mRNA expression.

17. The method of claim 15, comprising selecting cells from the cell population produced by steps a), b) or c) for expression of inhibitor of DNA binding 2 (Id2) or retinoid-related orphan receptor gamma (Rorc) protein or mRNA.

18. The method of claim 15, 16 or 17, further comprising purifying, isolating or enriching the LTi cells by cell sorting.

19. The method of claim 15, wherein the human donor is about from 1 hour to 100 years in age.

20. An in vitro or ex vivo method of expanding or increasing numbers of LTi cells, comprising contacting the cells of claim 1 or 2 with human embryonic kidney-293T (HEK-293T, ATCC CRL-11268), NHDF (normal human dermal fibroblasts) or HT-29 (colonic epithelial tumor cell line, ATCC HTB-38) cells, or a human embryonic kidney-293T (HEK-293T, ATCC CRL-11268), NHDF (normal human dermal fibroblasts) or HT-29 (colonic epithelial tumor cell line, ATCC HTB-38) cell conditioned medium under conditions that expand or increase numbers of LTI cells.

21. A method of identifying a compound that increases or decreases proliferation, growth, survival, or viability of LTi cells, comprising contacting the LTi cells of claim 1 or 2 with a test compound, and determining whether the test compound increases or decreases proliferation, growth, survival or viability of LTi cells.

22. The method of claim 21, wherein the test compound is a growth factor, a cytokine, an antibody, or a drug.

23. The method of claim 21, wherein the test compound comprises a fraction of a cell culture supernatant.

24. The method of claim 23, wherein the cell culture supernatant comprises a HEK-293T, NHDF or HT-29 cell culture supernatant.

25. The method of claim 21, wherein the test compound comprises a library of compounds.

26. The method of claim 25, wherein the library comprises a protein or antibody library.

27. The method of claim 21, wherein the LTi cells do not express or express a non-functional signaling protein.

28. The method of claim 27, wherein the signaling protein comprises a ligand, receptor, growth factor, or a cytokine.

29. The method of claim 21, wherein the LTi cells are mammalian.

30. A method of treating a subject in need of or that would benefit from LTi cells, comprising administering the LTi cells of claim 1 or 2 to a subject thereby providing the subject with the LTi cells.

31. The method of claim 30, wherein the subject has a viral infection or compromised lymphoid structures.

32. The method of claim 30, wherein the subject has a chronic viral infection.

33. The method of claim 30, wherein the subject is immunocompromised or immunosuppressed.

34. The method of claim 30, wherein the subject is infected with a herpesvirus or HIV.

35. The method of claim 30, wherein the LTi cells are autologous to the subject.
